(19) United States
(12) Patent Application Publication
(10) Pub. No.: US 2025/0383295 A1
(43) Pub. Date: Dec. 18, 2025

(54) PEARL GRADING INSTRUMENT AND METHODS

(71) Applicant: Gemological Institute of America, Inc.)GIA), Carlsbad, CA (US)

(72) Inventors: Zhen Wang, Carlsbad, CA (US); Yong Liu, Carlsbad, CA (US); Kevin De Leon, Carlsbad, CA (US); rj Pisani, Carlsbad, CA (US); Mehdi Toosi, Carlsbad, CA (US)

(21) Appl. No.: 19/242,625

(22) Filed: Jun. 18, 2025

Related U.S. Application Data
(60) Provisional application No. 63/661,411, filed on Jun. 18, 2024.

(51) Int. Cl. G01N 21/88; G01N 21/25; G01N 21/95; G01N 33/00

(52) U.S. Cl. CPC G01N 21/8806; G01N 21/253; G01N 21/951; G01N 33/389; G01N 2021/8816; G01N 2021/8841; G01N 2201/062; G01N 2201/0634

(57) ABSTRACT

Systems and methods for capturing images of pearls and determining characteristic grades of the pearls are provided. A top digital camera can capture a first digital image of the pearl. A stage can support the pearl and can move in any of an X/Y/Z direction. A concave diffuser can direct diffused light on one side of the pearl, and a side LED arrangement can illuminate the concave diffuser, the stage, and a second side of the pearl. A side digital camera can capture a second digital image of the pearl. A computer can obtain the digital images of the pearl and perform analysis to determine one or more characteristic grades for the pearl.

### CROSS-REFERENCE TO RELATED APPLICATION(S)

The present application claims priority to U.S. Provisional Patent Application No. 63/661,411, titled “PEARL GRADING INSTRUMENT AND METHODS,” and filed Jun. 18, 2024, the entirety of which is incorporated by reference herein.

### FIELD

The field includes systems and methods for analyzing and grading pearls using digital image capture and image analysis.

### BACKGROUND

Pearls have a long history as a jewelry material. Various aspects of a pearl can be graded, such as dimensions of the pearl, a quality of the pearl or an origin of the pearl. Many methods for grading pearls rely on a human operator using visual evaluation under a light box.

However, even for experienced human graders, variations in grading results are expected. Further, many pearl samples can be received as part of a strand, leading to manual grading of the pearl strand by human graders being time consuming and resource-intensive. It would be beneficial to the grading industry if an automated system and method could be implemented to grade pearls and ensure constant grading results based on digital instrumentation.

### SUMMARY

Systems for imaging and grading a pearl, may include a top digital camera configured to capture digital images, a top camera lens configured to magnify light passing to the top digital camera, the top camera lens including an LED (the “top LED”) arrangement. The system can also include a computer in communication with the top digital camera to capture digital images and receive the digital image data, a stage configured to support the pearl, wherein the stage is configured with motors to move the stage in an X, Y, and Z direction, a top-side LED (the “side LED”) arrangement configured to illuminate the stage and one side of the pearl supported on it, a concave diffuser configured to direct diffuse light on a second side of the pearl on the stage. The diffuser can be made of Polytetrafluoroethylene. The system can also include a side digital camera configured to capture digital images.

Systems and methods for capturing images of pearls and determining characteristic grades of the pearls are provided. A top digital camera can capture a first digital image of the pearl. A stage can support the pearl and can move in any of an X/Y/Z direction. A concave diffuser can direct diffused light on one side of the pearl, and a side LED arrangement can illuminate the concave diffuser, the stage, and a second side of the pearl. A side digital camera can capture a second digital image of the pearl. A computer can obtain the digital images of the pearl and perform analysis to determine one or more characteristic grades for the pearl.

In a first example embodiment, a system for imaging and grading a pearl is provided. The system can include a top digital camera configured to capture a first digital image. The system can also include a top camera lens configured to magnify light passing to the top digital camera, the top camera lens including a top light-emitting diode (LED) arrangement.

The system can also include a stage configured to support the pearl. The stage can be configured with stage motors to move the stage in any of an X, Y, and Z direction. The system can also include a concave diffuser configured to direct diffuse light on one side of the pearl on the stage. The system can also include a side LED arrangement configured to illuminate the concave diffuser, the stage, and a second side of the pearl supported on the stage.

The system can also include a side digital camera configured to capture a second digital image. The system can also include a side camera lens configured to magnify light passing to the side digital camera. The system can also include a computer in communication with the top digital camera and the side digital camera to receive the first digital image and the second digital image. The computer can be configured to determine one or more characteristic grades for the pearl based on the first digital image and the second digital image captured by the top digital camera and side digital camera.

In some instances, the side LED arrangement produces light with 5300 K color temperature with wavelengths ranging from around 350 to 1000 nanometers (nm).

In some instances, the stage motors are in communication with the computer, and wherein the computer is configured to analyze the received digital images from the top digital camera and the side digital camera and send instructions to the stage motors to move the stage to focus and align the pearl with the top digital camera and the side digital camera.

In some instances, the stage includes rotation stage motors configured to allow rotating of the stage about an x and/or y axis.

In some instances, top LED arrangement and the side LED arrangement are in communication with the computer, wherein the computer is configured to send instructions to top LED arrangement and side LED arrangement to turn on or off and to configure an arrangement of light produced by top LED arrangement and side LED arrangement.

In some instances, the computer is configured to receive the digital images from the top digital camera and the side digital camera and determine a size grade or a roundness grade of the pearl based on boundary detection of pixels from received digital images.

In some instances, the computer is configured to receive the digital images from the top digital camera and determine any of the one or more characteristic grades that include any of: a luster grade, a body color grade, and an overtone grade of the pearl based on the received digital images.

In some instances, the computer is configured to turn off the side LED arrangement, turn on the top LED arrangement, and cause the capture of the first digital image by the top digital camera to determine a luster grade of the pearl by:
calculating a max luminance (Lumi_max) and a background luminance (Lumi_bg) from the first digital image;
locating a bright spot based on the Lumi_max and the Lumi_bg at a threshold of luminance at:

$$Th = ( Lumi\_max + Lumi\_bg ) ⋆ 0.5 ;$$

and
calculating a size of the bright spot based on boundary detection of pixels in the first digital image.

In some instances, the computer is configured to turn off the top LED arrangement, turn on the side LED arrangement, cause the capture of the first digital image by the top digital camera and apply a contrast filter to the first digital image to find three zones comprising: a boundary zone, a diffuse light zone, and a direct light zone, and analyze the three zones to determine body color and overtone, wherein the applying the contrast filter to find three zones includes:
creating a mask of the pearl as M-all;
creating a dark region as M-dark, a bright region as M-bright based on a histogram
creating an inner circle mask as M-inner based on the dark region; and
creating the three zones based on M-all, M-dark, M-bright, and M-inner, wherein the boundary zone is between M-all and M-inner, wherein the direct light zone is M-bright, wherein the diffuse light zone is M-dark.

In some instances, the computer is configured to, for white and light-colored pearls, analyze the boundary zone and the diffuse light zone for a body color grade, and analyze the direct light zone for a overtone grade.

In some instances, the pearl comprises a dark-colored pearl, the computer is configured to analyze the boundary zone and the direct light zone for an overtone grade, and the diffuse light zone for a body color grade.

In some instances, the computer is configured to perform a color analysis on the boundary zone, the diffuse light zone, and the direct light zone, for each zone calculate a mean value of Hue, by:
converting RGB to HSV on each pixel, H: hue in range 0°˜360°;
converting for each Hue value to (a*, b*) plane by:

$${a}^{*} = cos ( h )$$

$${b}^{*} = sin ( h )$$

calculating mean values a̅*̅ and b̅*̅;
calculating the mean value of Hue of each zone by:

$$\overline{h} = a tan ( \overline{{b}^{*}} / \overline{{a}^{*}} ) ;$$

and
evaluating the body color and overtone of the pearl based the mean values of Hue of each zone.

In another example embodiment, a method for imaging and grading a pearl. The method can include sending, by a computer, a first instruction to a side light-emitting diode (LED) to cause illumination of a first side of the pearl with direct light from the side LED. The method can also include causing, by the computer, illumination of a second side of the pearl by a concave diffuser configured to diffuse light from the side LED on the second side of the pearl.

The method can also include causing a capture of a first digital image of the pearl by a side digital camera. The method can also include receiving the first digital image from the side digital camera. The method can also include detecting a top surface of each pearl from the first digital image captured by the side digital camera. The method can also include aligning the top surface of each pearl to the side digital camera.

The method can also include causing a capture of a second digital image of the pearl by a top digital camera, the second digital image including the pearl illuminated on the first side by the direct LED light and the diffused LED light on the second side. The method can also include receiving the second digital image from the top digital camera. The method can also include calculating one or more characteristic grades for the pearl based on the received digital images from the top digital camera and side digital camera.

In some instances, the method can include sending, by the computer, an instruction to motors on a stage configured to support the pearl, wherein the stage is configured with motors to move the stage in an X, Y, and Z direction.

In some instances, the method can include determining, by the computer, any of a size grade and a roundness grade of the one or more characteristic grades for the pearl based on boundary detection of pixels in the received digital images.

In some instances, the method can include determining, by the computer, any of a luster grade, a body color grade, and an overtone grade of the one or more characteristic grades for the pearl based on the received digital images.

In some instances, the method can include
turning off the side LED;
turning on a top LED; and
causing the capture of the second digital image by the top digital camera to determine a luster grade by calculating a max luminescence number displayed in the second digital image by:
calculating a max luminance (Lumi_max) and a background luminance (Lumi_bg) from the digital image;
locating a bright spot based on the Lumi_max and the Lumi_bg at a threshold of luminance at:

$$th = ( Lumi\_max + Lumi\_bg ) * 0.5 ;$$

calculating a size of the bright spot based on boundary detection of pixels in the second digital image.

In some instances, the method can include
turning off a top LED;
turning on the side LED; and
applying a contrast filter to the second digital image to find three zones: a boundary zone, a diffuse light zone, and a direct light zone, and analyzing the three zones to determine a body color and an overtone of the pearl, wherein the applying the contrast filter to find three zones includes:
creating a mask of the pearl as M-all;
creating a dark region as M-dark, a bright region as M-bright based on histogram analysis;
creating an inner circle mask M-inner based on dark region; and
creating 3 zones based on M-all, M-dark, M-bright, and M-inner, wherein the boundary zone is between M-all and M-inner, wherein the direct light zone is M-bright, and wherein the diffuse light zone is M-dark.

In some instances, the method can include
for white and light-colored pearls, analyzing, by the computer, the boundary zone and the diffuse light zone for a body color grade, and analyzing the direct light zone for an overtone grade; and
for dark-colored pearls, analyzing, by the computer, the boundary zone and the direct light zone for an overtone grade, and analyzing the diffuse light zone for a body color grade.

In some instances, a body color grade and an overtone grade is determined by color analysis on the boundary zone, the diffuse light zone and the direct light zone, for each zone calculating a mean value of Hue, by,
converting of RGB to HSV on each pixel, H: hue in range 0°˜360°;
converting for each Hue value to (a*, b*) plane by:

$${a}^{*} = cos ( h )$$

$${b}^{*} = sin ( h )$$

calculating mean values a̅*̅ and b̅*̅;
calculating the mean value of Hue of zone by:

$$\overline{h} = a tan ( \overline{{b}^{*}} / \overline{{a}^{*}} ) ;$$

and
evaluating the body color and the overtone of the pearl based the mean values of Hue for each zone.

### BRIEF DESCRIPTIONS OF THE DRAWINGS

For a better understanding of the embodiments described in this application, reference should be made to the Detailed Description below, in conjunction with the following drawings in which like reference numerals refer to corresponding parts throughout the figures.

FIG. 1 is a front facing illustration of an example imaging system in accordance with certain aspects described herein;

FIG. 2A is a first side view of an example imaging system in accordance with certain aspects described herein;

FIG. 2B is a second side view of an example imaging system in accordance with certain aspects described herein;

FIG. 2C is a schematic of a diffuser example, in accordance with certain aspects described herein;

FIG. 3 is a top facing illustration of an example imaging system in accordance with certain aspects described herein;

FIG. 4 is an example flow chart of a grading method that may be used to grade pearls, using certain aspects described herein;

FIG. 5 is an example flow chart of a grading method that may be used to grade pearls, using certain aspects described herein;

FIG. 6 is an example pearl illumination grading example that may be used to grade pearls, using certain aspects described herein;

FIG. 7 is an example flow chart of a grading method that may be used to grade pearls, using certain aspects described herein;

FIG. 8 is an illustration of a pearl detail for an example imaging system in accordance with certain aspects described herein;

FIG. 9 is a diagram of an example networked system in accordance with certain aspects described herein; and

FIG. 10 is a diagram of an example computer system in accordance with certain aspects described herein.

### DETAILED DESCRIPTION

Reference will now be made in detail to embodiments, examples of which are illustrated in the accompanying drawings. In the following detailed description, numerous specific details are set forth in order to provide a sufficient understanding of the subject matter presented herein. But it will be apparent to one of ordinary skill in the art that the subject matter may be practiced without these specific details. Moreover, the particular embodiments described herein are provided by way of example and should not be used to limit the scope of the particular embodiments. In other instances, well-known data structures, timing protocols, software operations, procedures, and components have not been described in detail so as not to unnecessarily obscure aspects of the embodiments herein.

### Overview

Systems and methods described here may be used to illuminate, view, image, and analyze pearls including automatically assigning a grade based on the images.

Hardware, software, and methods are described that can be used to digitally image pearls under specific lighting conditions as well as to apply imaging processing technology to the captured images in order to grade and analyze pearls. This automated, digital system can result in more consistent grading results than human graders and allow for better record keeping with image and grade storage.

By automating the image capture and analysis under consistent conditions this system can take the human variation factor out of the pearl grading and provide better, more reliable, and more consistent results.

### Hardware Examples

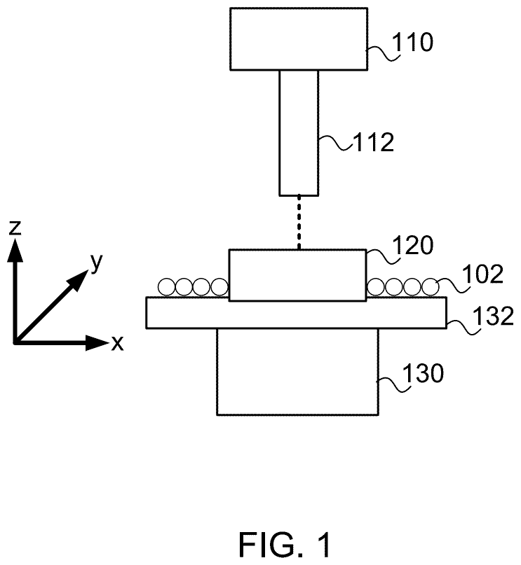

Systems described here may include a collection of component parts assembled into a unit that may be used by a human operator or by an automated computerized system to utilize specialized lighting arrangements to illuminate pearl sample(s), digitally image them, and analyze the digital images for grading and storage purposes. An example of such hardware is shown in FIG. 1 from the front, 2 from the side and 3 from the top.

As shown in the example of FIG. 1, illustrating an example hardware arrangement of a front view of the hardware arrangement described and used here. In FIG. 1, the pearl 102 under evaluation is shown alongside an array of other samples. In some examples, the pearls 102 are in a strand such as a necklace and positioned on the stage as described. The pearl 102 under evaluation is the one lined up with the instruments in order to correctly illuminate and digitally image it. The array of other pearls may be queued such that each in turn may be placed in the stage section where the lighting and instruments are focused, but close enough such that they may be quickly moved there and back. In some examples, a queue of pearls may be moved down the line such that one at a time comes under the focal point of the stage for illumination and image capture and then moved down to the next and so on until all pearls in the queue have been illuminated and imaged.

The pearl stage may be configured to rest securely within a recessed nest on an aluminum plate. This arrangement may keep both the pearls and the stage from drastically shifting its position as it is being moved during the process of illumination and imaging. The movement of the pearl stage may be achieved by using a combination of one manual and one automatic positioning stage. In some examples, it may be automatic and not manual.

The aforementioned aluminum plate on which the pearl stage rests may be mounted to the top of a motorized linear guide. In some examples, there may be a 110-millimeter stroke to the mount. The linear guide itself may be mounted to the top of a manual or automated three-axis stage that can be positioned using three different knobs for manual and three different motors for automated.

In examples with a manual stage, the system provides adjustment for the position of the central focal point of the pearl stage in the X and Y directions relative to the view of the top digital camera. Additionally, the manual stage has the capability of adjustment in the Z direction which allows for image focusing. The motorized linear guide may be controlled using a computer in communication with motors and computerized software that sends and receives commands to the motors to provide single-axis travel of the pearl stage under the camera. This arrangement may enable the capability of having a queue of pearls on the pearl stage moved under the central focal point automatically, one by one, for illumination and imaging.

In some examples, such queues of pearls may be loaded into the system for evaluation which may save time over systems that must individually load one pearl at a time. Such a queuing system may include packs or stacks of pearls loaded into the system as described.

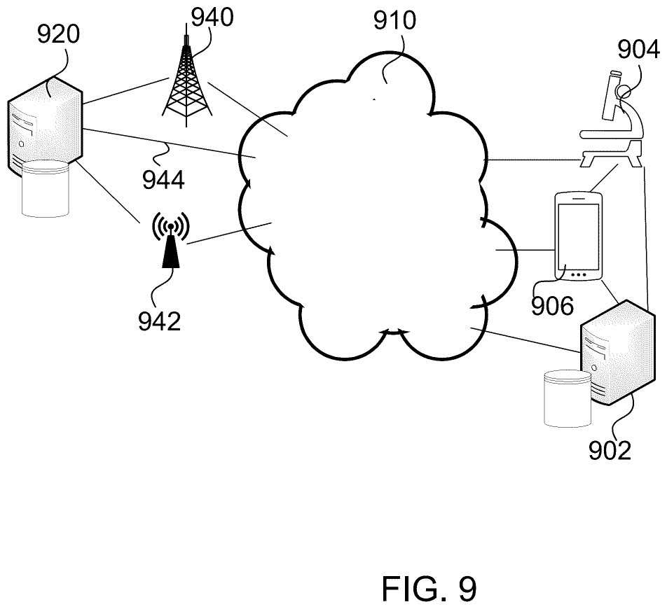

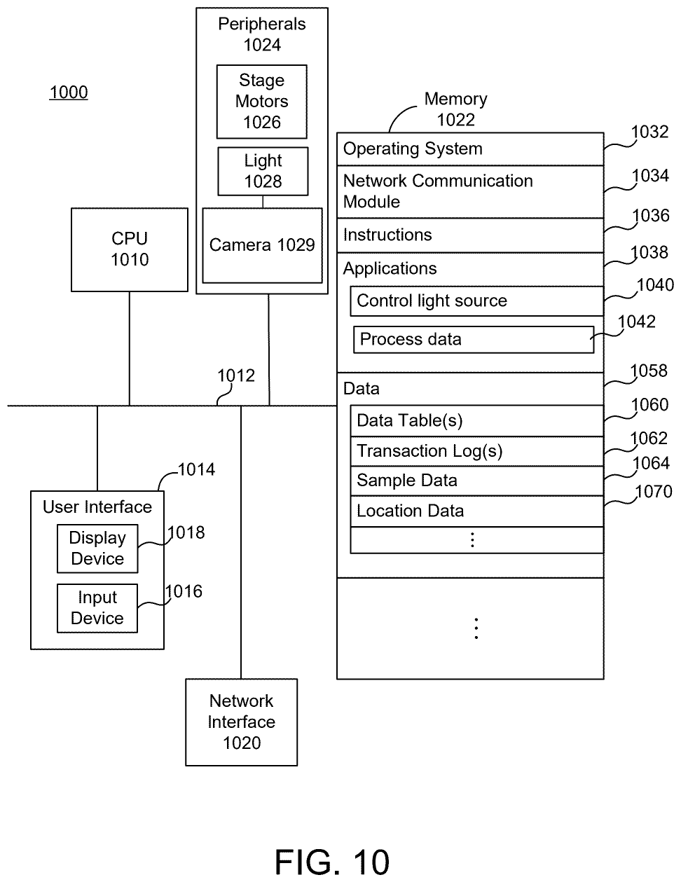

Once loaded into the system and placed in the central focal point of the stage, the pearl 102 under analysis may come in view of a top digital camera 110 aimed at the pearl(s) 102 on the stage. The top digital camera 110 is shown with a top imaging lens 112 attached or in communication with the top digital camera 110. In some examples, the imaging lens 112 includes its own built-in LED array. Such an LED system may be in communication with the computer(s) as described herein. In some examples, the top digital camera 110 is a GS3-U3-515SC-C(FLIR). In some examples this camera has infra-red capabilities. In some examples, the top imaging lens is FXL-0.19x-110D-CESF. Other types of cameras and lens may be used for this setup, calibration may be needed after modification. Such a digital camera may be arranged in communication with a computer or include computer component parts within it. Such a computer may be described in FIG. 10. Such a network and computer components the camera 110 may be a part of is described in FIG. 9.

The pearl(s) 102 under evaluation are shown on a motion stage 130 with top stage 132 where they are supported. The motion stage 130 may be configured with electric motors to move the stage in X, Y, and/or Z direction in relation to the camera 110 arrangement. In some examples, the motion stage 130 is MAXYZ-90L-H (Optics Focus). In some examples, such stage motors are in communication with the computer and/or include computer component parts described in FIG. 10 and networked in FIG. 9.

In some examples, this motion stage 130 may allow the queue of pearls 102 to incrementally move in the X direction one at a time into the center of the system for illumination and imaging by the camera(s). In some examples, the stage 130 may move in the Z direction to allow for better focusing of the camera(s) on the pearls. In some examples, the stage 130 may move in the Y and/or X direction(s) to allow for alignment of the pearls 102 to a field of view of the camera(s). In some examples, the stage may be composed of motorized x-y and manual z-stage. In such examples, the z axis only needs to be adjusted once for focusing, when the sample was first loaded to the system.

Also shown in the hardware arrangement is a diffuser 120 configured to diffuse light onto or around the pearl(s) under evaluation 102. Such a diffuser diffuses light from side LED light sources 118 to illuminate the stage 132 and the pearls 102 as described herein.

In some examples, the diffuser 120 is made of virgin Polytetrafluoroethylene (Teflon). In some examples, other polymers may be used. In some examples, the diffuser may be 3D printed. In some examples, the diffuser maybe machine milled. In some examples, the diffuser may be white in color.

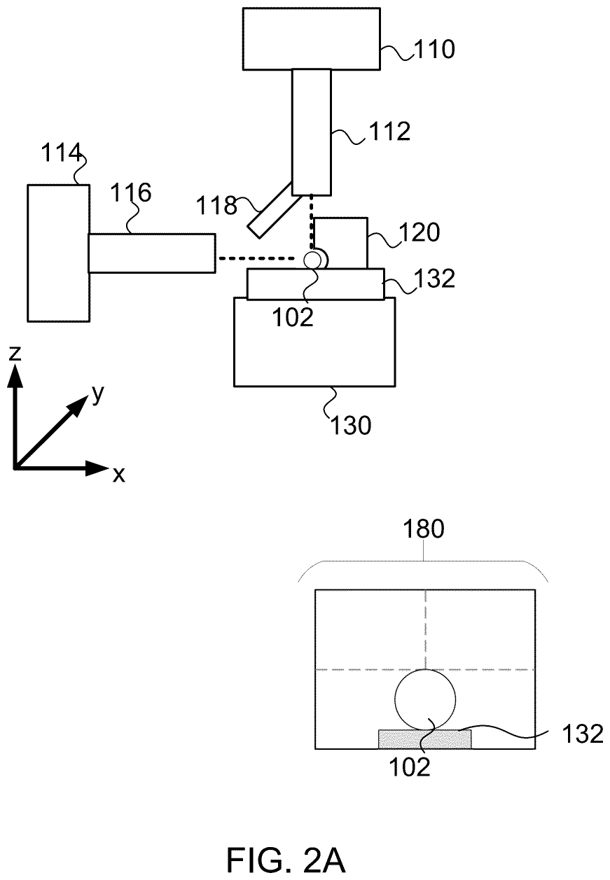

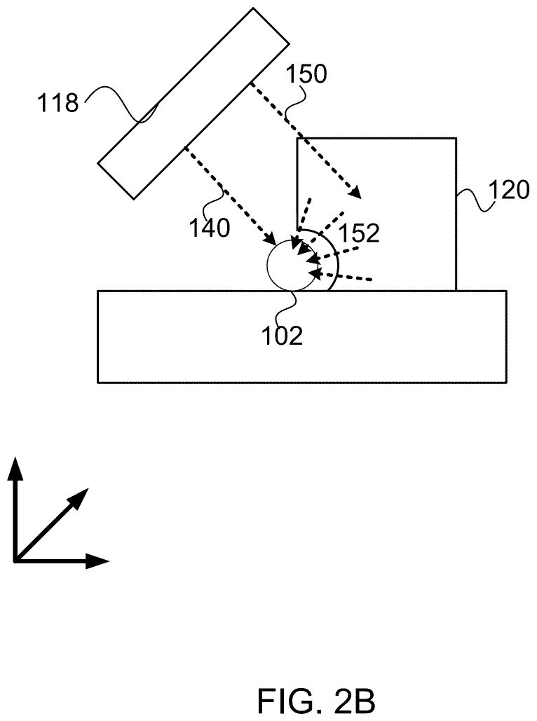

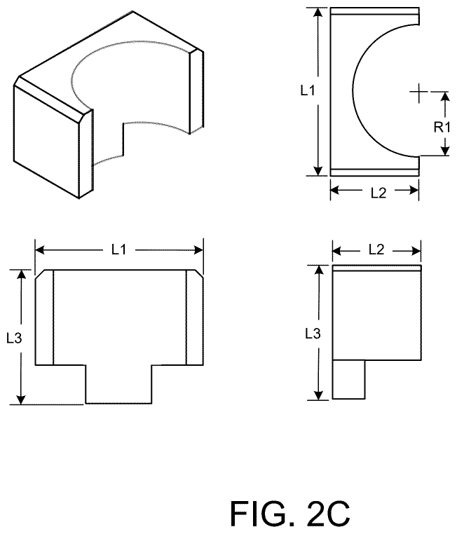

FIG. 2A is an illustration of example side views of the example hardware system as shown in FIG. 1. The FIG. 1 arrangement shows the same parts as FIG. 1 with the addition of a second camera aimed from the side 114 with its own imaging lens 116. In some examples, the side digital camera is GS3-U3-515SC-C(FLIR). In some examples, this is an infra-red capable camera. In some examples, the side lens is a TC23024 (Opto-Engineering). In some examples, such side digital camera 114 is in communication with the computer and/or include computer component parts described in FIG. 10 and networked in FIG. 9.

Then the sample was firstly loaded into the system, top surface of each pearl 102 may be aligned to the center of side digital camera 114 as shown in the close up detail 180. During the measurement, pearl size and roundness information can be captured by the side digital camera 114 and top digital camera 110. Such image capture may be used to grade the roundness of a pearl 102 based on a silhouette or outline or boundary analysis.

Also shown is a side LED light 118 arrangement angled to project light toward the top stage 132 and pearl(s) 102 on the top stage 132. In some examples, this side LED is 5300 K temperature with wavelengths from 350-1000 nm. In some examples, such side LED lighting 118 is in communication with the computer and/or include computer component parts described in FIG. 10 and networked in FIG. 9. In some examples, there is a built-in top LED in the top camera lens 112.

As can be seen from FIG. 2A side view, the diffuser 120 may include a cutout section where the pearls are partially surrounded by the diffuser to provide a lighting environment that illuminates a wider angle than as from a point source of light.

FIG. 2B is a close-up detail view of the LED 118 directing light beams 140, 150 directly on the pearl 102 from one side and also the LED 118 directing light beams into the diffuser 120 which diffuses light 152 back onto the pearl 102 from the diffuser side 120.

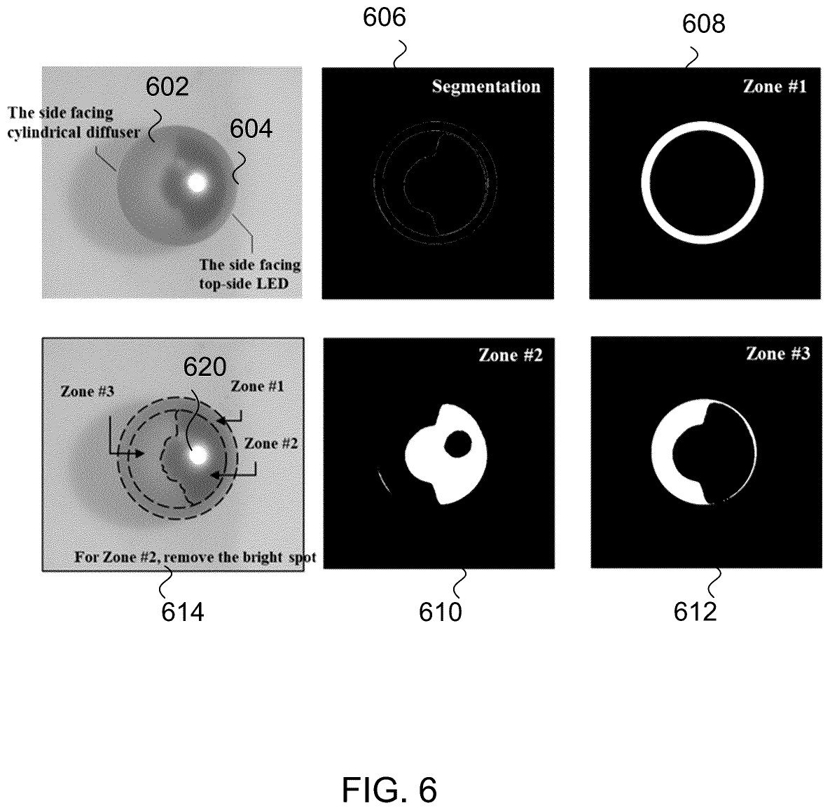

In such a way, as can be seen from FIG. 2B, the diffuser 120 is able to redirect diffuse light 152 from the LED 118 to the pearl 102 and because of its shape, allow direct LED light 140 to illuminate the other side of the pearl 102. This allows for a multi illuminated pearl as shown and described in FIG. 6.

FIG. 2C shows example diagrams of a diffuser as described herein (120 from FIG. 2A) with the width L1 of around 3 inches wide, the width L2 of around 1.57 inches deep, the height L3 of around 2.36 inches tall, and with a semi-circular cutout having a radius RI of around a 1.18 inch radius. These example dimensions are not intended to be limiting and could be any dimensions to fit around the queue of pearls and diffuse light as described.

For rough dimension of location of side LED, regarding the center of the LED as the point of reference, the “X” distance (distance from center of LED 118 in FIG. 2A to the front edge of the diffuser 120) may be about 93 mm, or 3.66 inches. In some examples, the distance X may be between 3 and 4 inches. In some examples, the distance X may be between 2 and 5 inches. The “Y” distance from (distance from center of LED 118 to the bottom surface of diffuser/top surface of the pearl stage 132) may be about 76.2 mm, 3 inches. In some examples, the Y distance may be between 2.5 and 3.5 inches, In some examples, the Y distance may be between 2 and 4 inches.

Other types of pearl sample holders may be used for this hardware setup, alone or in combination with that shown in FIGS. 1, 2 and 3, for example, a round shape holder maybe used for long pearl strand, special mold in stage maybe used for mounted pearl jewelry pieces. In some examples, a white background for color calibration purposes may be useful for the diffuser.

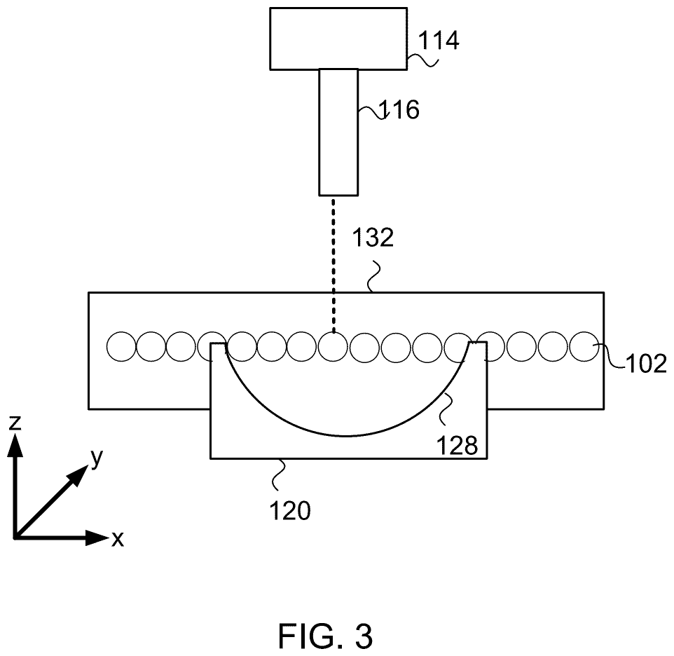

FIG. 3 shows a top-down view of the stage system showing the top stage 132 and queue of pearl(s) 102 along with the diffuser 120. As can be seen from the top-down view in FIG. 3, the diffuser includes a concave section 128 that is configured to diffuse light from the LED light source (118 from FIGS. 2A and 2B) to diffuse the light and direct it toward the pearl queue but from this 180 degree angle that surrounds the pearls and not from a direct pin source. In some examples, this concave section 128 may have a cylindrical shape.

In some examples, two different lightning environments may be utilized for this hardware setup and engineered to aid in analysis of particular qualities such as pearl body color, overtone, luster, size and sample roundness grading.

In such examples, digital image capture from the camera(s) and advanced image processing technology may utilize zone segmentation generated by above mentioned lightning environments to obtain body color and overtone from a single digital image, as described.

FIG. 6 shows illustrations of the pearl and images taken by the system for measuring Body Color and Overtone as described herein. In such an example the pearl would be placed in the system on the stage and this image in FIG. 6 would be taken from the top-down camera (110 in FIG. 1-3).

The image of the pearl 602 shows an image taken with one side of the pearl facing and illuminated by the cylindrical diffuser (120 from FIGS. 1-3) and another side of the pearl 604 facing and illuminated by the top side LED (118 from FIGS. 1-3) directly. This image is analyzed using segmentation 606 of the captured digital image to break out the different zones in the image of the pearl. This image analysis is done by a computer in communication with the camera to process and analyze the pixels in the digital image data. The representations shown in FIG. 6 may or may not be displayed to a user operator and may be stored as image data for other analysis, matching, or for storage purposes. The examples showing black and white images may be after a contrast filter is applied to the image data to enhance the differences in the illumination zones and make them binary options instead of gradient tones for easier image analysis. In some examples, a threshold is set for the pixel value in order to apply this contrast filter. In some examples, this threshold is a brightness value of B1, in some examples, a brightness value of B2.

In some examples, alone or in combination, digital images may be directly captured from the top digital camera 110 for colored images. The black and white images may be generated by the computer as segmentation results after image processing by computer software, the only purpose is to separate the colored pearl image into different zone and perform post color analysis. The detailed segmentation methods may include, dividing the Pearl segmentations to create 3 zones:
1. Create mask of the pearl M-all
2. Based on histogram analysis, create the dark region M-dark, bright region M-bright
3. Create inner circle mask M-inner based on dark region
4. Create 3 zones based on M-all, M-dark, M-bright, and M-inner

The image is analyzed by the computer to be broken into zones: Zone 1 608, Zone 2 610 and Zone 3 612. The image 614 shows the initial image and all the zones overlayed on top in a composite image.

This composite image may be used by the system to remove the bright spot 620 that shows up as a reflection on the pearl. These images can be used to grade body color and overtone of the pearl. Further, pixel analysis by the software may be made. For example, Color analysis on zone #1, zone #2 and zone #3: For each zone calculate the mean value of Hue following the steps below:
RGB to HSV conversion on each pixel, H: hue in range 0°˜360°
For each Hue value, convert to (a,b*) plane by

$${a}^{*} = cos ( h )$$

$${b}^{*} = sin ( h )$$

Calculate the mean values a̅*̅ and b̅*̅
Calculate the mean value of Hue of zone by

$$\overline{h} = a tan ( \overline{{b}^{*}} / \overline{{a}^{*}} )$$

The body color and overtone of pearl are evaluated based the mean values of Hue of zone #1, 2 and 3.

### Method Steps for Pearl Grading

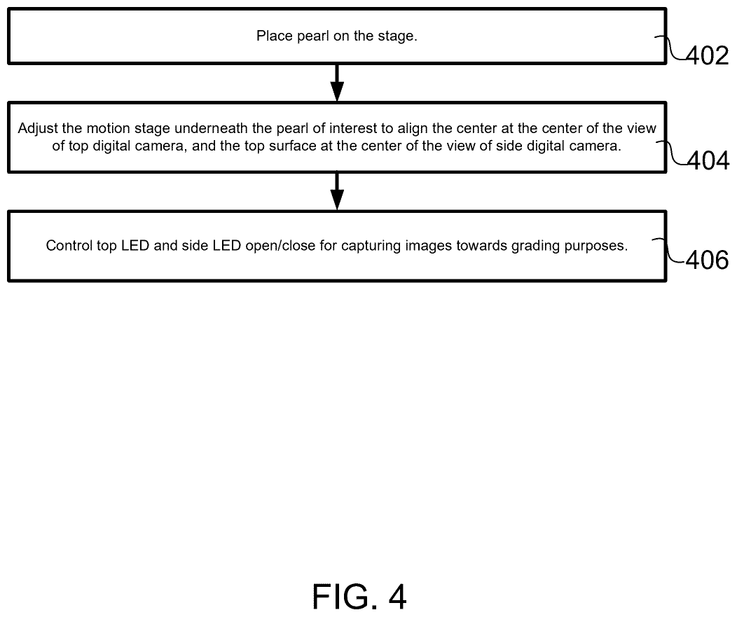

FIG. 4 includes a flow chart showing method steps used to capture and analyze images of pearls using the systems described herein. As shown in FIG. 4, at 402, one or more pearls can be placed on stage.

At 404, the motion stage can be adjusted to place the pearl of interest at the center of view of both top and side digital cameras. At 406, the top and side LED can be controlled for capturing images for grading purposes. As described in FIG. 1-3, the stage motors in the stage may be used to move the stage. In some examples, such stage motors may be in communication with a computer as described in FIG. 10 and/or a network as described in FIG. 9.

### Body Color and Overtone Examples

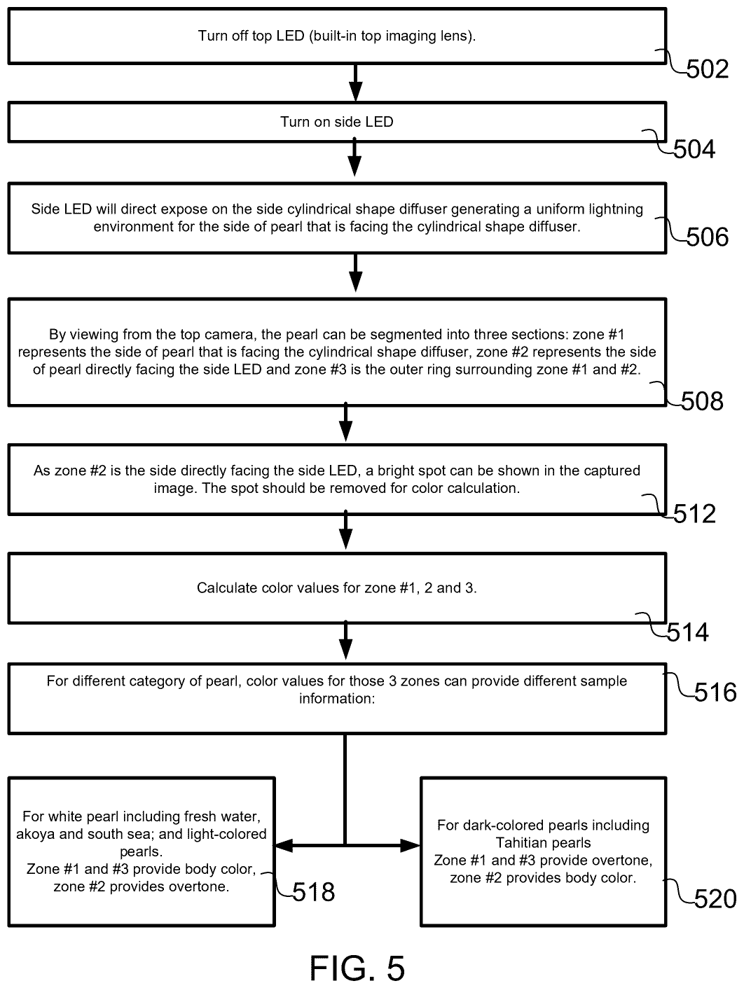

FIG. 5 includes a flow chart showing method steps used capture and analyze images of pearls using the systems described herein to measure body color and overtone. At 502, a top LED from the built-in top imaging lens in FIG. 2A can be turned off. At 504, a side LED can be turned on to emit LED light.

At 506, the side LED may direct expose on the side cylindrical shape diffuser generating a uniform lightning environment for the side of pearl that is facing the cylindrical shape diffuser. At 508, by viewing from the top digital camera, the pearl can be segmented into three sections: zone #1 represents the side of pearl that is facing the cylindrical shape diffuser, zone #2 represents the side of pearl directly facing the side LED and zone #3 is the outer ring surrounding zone #1 and #2. This is shown and described in more detail in FIG. 6.

At 512, as zone #2 is the side directly facing the-size LED, a bright spot can be shown in the captured image. The spot can be removed for color calculation.

At 514, color values can be captured for zone #1, 2 and 3. Next, at 516, for different categories of pearls, color values for those 3 zones provides different sample information.

At 518, for white pearls including fresh water, akoya and south sea; and light-colored pearls. Zones #1 and #3 can provide body color, and zone #2 can provide overtone.

At 520, for dark-colored pearls including Tahitian pearls and other dark-colored pearls zones #1 and #3 can provide overtone, and zone #2 can provide body color.

Computer software may be used for further color analysis such as, color analysis on zone #1, zone #2 and zone #3:

For each zone calculate the mean value of Hue following the steps below:
RGB to HSV conversion on each pixel, H: hue in range 0°˜360°
For each Hue value, convert to (a*,b*) plane by

$${a}^{*} = cos ( h )$$

$${b}^{*} = sin ( h )$$

Calculate the mean values a̅*̅ and b̅*̅
Calculate the mean value of Hue of zone by

$$\overline{h} = a tan ( \overline{{b}^{*}} / \overline{{a}^{*}} )$$

The body color and overtone of pearl are evaluated based the mean values of Hue of zone #1,2 and 3.

### Luster Examples

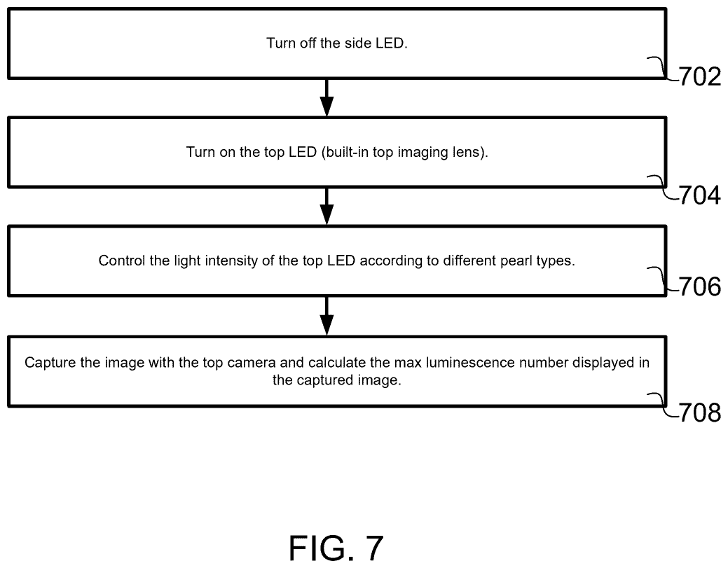

FIG. 7 shows an example flow chart on how to use the system to measure pearl luster.

At 702, a side LED can be turned off (118 in FIG. 2A). At 704, a top LED (built-in top imaging lens) can be turned on.

At 706, the light intensity of the top LED can be controlled according to different pearl types. For each type of pearl, the number is constant. At 708, the image can be captured with the top digital camera and calculate the max luminescence number displayed in the captured digital image with a computer. The number may be compared in the database for that specific category of pearl samples and provide a luster grading.

Luster analysis may be calculated by the computer by: capturing a digital image with fixed shutter time by the top digital camera, calculating the max luminance Lumi_max and background luminance Lumi_bg from the digital image, and calculating the area, width and length of the brightness spot of the pearl based on the threshold.

$$th = ( Lumi\_max + Lumi\_bg ) * 0.5$$

The calculated area, width and length can be used to evaluate the luster of pearl.

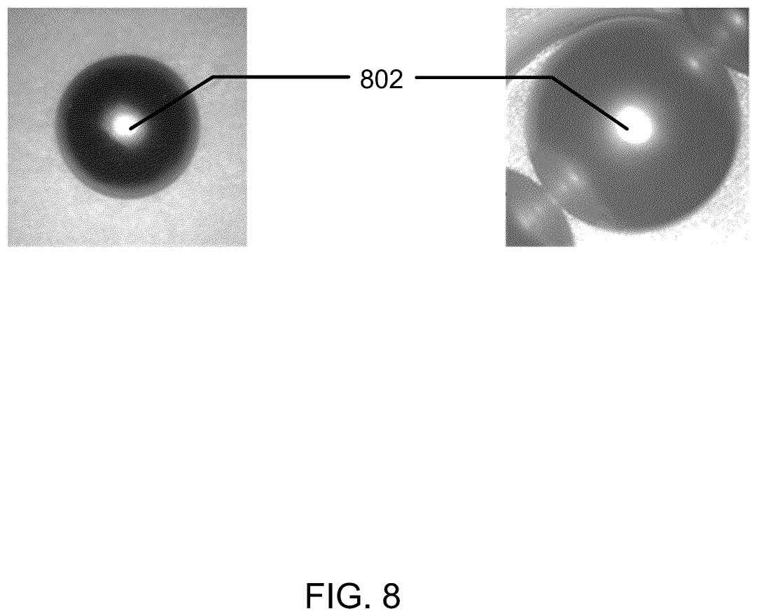

FIG. 8 shows an example image of the bright spot 802 illumination resulting from an image capture when the top LED is on and side LED is off.

### To Measure Size and Sample Roundness

Size and roundness may be calculated based on the top and size view images using the top and side digital cameras as described. Pearl strand may rotate 360 degrees in 20 degrees/step in for image saving. Additionally or alternatively, rotation angles other than 20 degrees may be used as rotation step. All images may be saved and analyzed by software towards the final calculation.

### Storage, Matching, and Grading

As can be seen from the description, the systems and methods described herein may be used to capture digital images and analyze the digital images in order to grade pearls under specific lighting conditions.

It may be beneficial to retain and store such image data along with correlated grades assigned by the system or a human who analyzes the data. Such storage may allow for later matching of a pearl sample to a previous auto grading event. Such storage may allow for data analysis over a large sample size. Such storage may allow for data sets to be compiled and utilized in training artificial intelligence systems for image analysis and auto grading purposes.

### Network Examples

Systems and methods here may utilize a networked computing arrangement as shown in FIG. 9. In FIG. 9, a computer 902 may be used to process the pixel data of the captured images of the camera, send and receive instructions to the stage motors, or send and receive other data such as sample location, identification information of the pearls, time and date, etc. The computer 902 used for these steps could be any number of kinds of computers such as those included in the camera itself, and/or another computer arrangement in communication with laboratory components 904 or camera computer components 906 including but not limited to a laptop, desktop, tablet, phablet, smartphone, or any other kind of device used to process and transmit digitized data.

Turning back to FIG. 9, the data captured for the pixelated image, calibration file, stone sample identifying information, location, and/or sample tilt from whichever computer 902 may be analyzed on a back end system instead of or in addition to a local computer. In such examples, data may be transmitted to a back end computer 930 and associated data storage 920 for saving, analysis, computation, comparison, or other manipulation. In some examples, additionally or alternatively, the transmission of data may be wireless (e.g., via wireless points 940, 942) by a cellular or Wi-Fi transmission with associated routers and hubs. In some examples, additionally or alternatively, the transmission may be through a wired connection 944. In some examples, additionally or alternatively, the transmission may be through a network such as the internet 910 to the back end server computer 902 and associated data storage 920. At the back end server computer 902 and associated data storage 920, the pixelated image data, calibration file, sample identification, sample location, time, date, and/or sample tilt may be stored, analyzed, compared to previously stored image data and/or image data for matching, identification, and/or any other kind of data analysis. In some examples, additionally or alternatively, the storing, analyzing, and/or processing of data may be accomplished at the computer 902 which is involved in the original image capture and/or data collection. In some examples, additionally or alternatively, the data storing, analyzing, and/or processing may be shared between the local computer 902 and a back end computing system 920. In such examples, networked computer resources 902 may allow for more data processing power to be utilized than may be otherwise available at the local computers 902. In such a way, the processing and/or storage of data may be offloaded to the compute resources that are available. In some examples, additionally or alternatively, the networked computer resources 920 may be virtual machines in a cloud or distributed infrastructure. In some examples, additionally or alternatively, the networked computer resources 920 may be spread across many multiple physical or virtual computer resources by a cloud infrastructure. The example of a single computer server 920 is not intended to be limiting and is only one example of a compute resource that may be utilized by the systems and methods described herein. In some examples, additionally or alternatively, artificial intelligence and/or machine learning may be used to analyze the image data from the samples, align the sample with the camera and/or focus the imaging camera for use with stage movement. Such systems may employ data sets to train algorithms to help produce better and better results of imaging of samples, alignment of samples, analysis of samples, identification of focused samples, stage movement, camera movement, and the like.

### Example Computer Devices

FIG. 10 shows an example computing device 1000 which may be used in the systems and methods described herein. In the example computer 1000 a CPU or processor 1010 is in communication by a bus or other communication 1012 with a user interface 1014. The user interface includes an example input device 1016 such as a keyboard, mouse, touchscreen, button, joystick, or other user input device(s). The user interface 1014 also includes a display device 1018 such as a screen. The computing device 1000 shown in FIG. 10 also includes a network interface 1020 which is in communication with the CPU 1010 and other components. The network interface 1020 may allow the computing device 1000 to communicate with other computers, databases, networks, user devices, or any other computing capable devices. In some examples, additionally or alternatively, the method of communication may be through WIFI, cellular, Bluetooth Low Energy, wired communication, or any other kind of communication. In some examples, additionally or alternatively, the example computing device 1000 includes peripherals 1024 also in communication with the processor 1010. In some examples, additionally or alternatively, peripherals include stage motors 1026 such as electric servo and/or stepper motors used for moving the stage for the sample analysis. In some examples peripherals 1024 may include camera equipment 1028, and/or lighting equipment 1029. In some examples computing device 1000 a memory 1022 is in communication with the processor 1010. In some examples, additionally or alternatively, this memory 1022 may include instructions to execute software such as an operating system 1032, network communications module 1034, other instructions 1036, applications 1038, applications to digitize images 1040, applications to process image pixels 1042, data storage 1058, data such as data tables 1060, transaction logs 1062, sample data 1064, sample location data 1070 or any other kind of data.

### CONCLUSION

As disclosed herein, features consistent with the present embodiments may be implemented via computer-hardware, software and/or firmware. For example, the systems and methods disclosed herein may be embodied in various forms including, for example, a data processor, such as a computer that also includes a database, digital electronic circuitry, firmware, software, computer networks, servers, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the embodiments or they may include a computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various machines may be used with programs written in accordance with teachings of the embodiments, or it may be more convenient to construct a specialized apparatus or system to perform the required methods and techniques.

Aspects of the method and system described herein, such as the logic, may be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, and so on).

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the descriptions have been specifically described herein, it will be apparent to those skilled in the art to which the description pertains that variations and modifications of the various implementations shown and described herein may be made without departing from the spirit and scope of the embodiments. Accordingly, it is intended that the embodiments be limited only to the extent required by the applicable rules of law.

The present embodiments can be embodied in the form of methods and apparatus for practicing those methods. The present embodiments can also be embodied in the form of program code embodied in tangible media, such as floppy diskettes, CD-ROMs, hard drives, or any other machine-readable storage medium, wherein, when the program code is loaded into and executed by a machine, such as a computer, the machine becomes an apparatus for practicing the embodiments. The present embodiments can also be in the form of program code, for example, whether stored in a storage medium, loaded into and/or executed by a machine, or transmitted over some transmission medium, such as over electrical wiring or cabling, through fiber optics, or via electromagnetic radiation, wherein, when the program code is loaded into and executed by a machine, such as a computer, the machine becomes an apparatus for practicing the embodiments. When implemented on a processor, the program code segments combine with the processor to provide a unique device that operates analogously to specific logic circuits.

The software is stored in a machine readable medium that may take many forms, including but not limited to, a tangible storage medium, a carrier wave medium or physical transmission medium. Non-volatile storage media include, for example, optical or magnetic disks, such as any of the storage devices in any computer(s) or the like. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or clectromagnetic signals, or acoustic or light waves such as those generated during radio frequency (RF) and infrared (IR) data communications. Common forms of computer-readable media therefore include for example: disks (e.g., hard, floppy, flexible) or any other magnetic medium, a CD-ROM, DVD or DVD-ROM, any other optical medium, any other physical storage medium, a RAM, a PROM and EPROM, a FLASH-EPROM, any other memory chip, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

The foregoing description, for purpose of explanation, has been described with reference to specific embodiments. However, the illustrative discussions above are not intended to be exhaustive or to limit the embodiments to the precise forms disclosed. Many modifications and variations are possible in view of the above teachings. The embodiments were chosen and described in order to best explain the principles of the embodiments and its practical applications, to thereby enable others skilled in the art to best utilize the various embodiments with various modifications as are suited to the particular use contemplated.

What is claimed is:

1. A system for imaging and grading a pearl, the system comprising:
a top digital camera configured to capture a first digital image;
a top camera lens configured to magnify light passing to the top digital camera, the top camera lens including a top light-emitting diode (LED) arrangement;
a stage configured to support the pearl, wherein the stage is configured with stage motors to move the stage in any of an X, Y, and Z direction;
a concave diffuser configured to direct diffuse light on one side of the pearl on the stage, a side LED arrangement configured to illuminate the concave diffuser, the stage, and a second side of the pearl supported on the stage;
a side digital camera configured to capture a second digital image;
a side camera lens configured to magnify light passing to the side digital camera; and
a computer in communication with the top digital camera and the side digital camera to receive the first digital image and the second digital image;
wherein the computer is configured to determine one or more characteristic grades for the pearl based on the first digital image and the second digital image captured by the top digital camera and side digital camera.

2. The system of claim 1, wherein the side LED arrangement produces light with 5300 K color temperature with wavelengths ranging from around 350 to 1000 nanometers (nm).

3. The system of claim 1, wherein the stage motors are in communication with the computer, and wherein the computer is configured to analyze the received digital images from the top digital camera and the side digital camera and send instructions to the stage motors to move the stage to focus and align the pearl with the top digital camera and the side digital camera.

4. The system of claim 1, wherein the stage includes rotation stage motors configured to allow rotating of the stage about an x and/or y axis.

5. The system of claim 1, wherein top LED arrangement and the side LED arrangement are in communication with the computer, wherein the computer is configured to send instructions to top LED arrangement and side LED arrangement to turn on or off and to configure an arrangement of light produced by top LED arrangement and side LED arrangement.

6. The system of claim 1, wherein the computer is configured to receive the digital images from the top digital camera and the side digital camera and determine a size grade or a roundness grade of the pearl based on boundary detection of pixels from received digital images.

7. The system of claim 1, wherein the computer is configured to receive the digital images from the top digital camera and determine any of the one or more characteristic grades that include any of: a luster grade, a body color grade, and an overtone grade of the pearl based on the received digital images.

8. The system of claim 1, wherein the computer is configured to turn off the side LED arrangement, turn on the top LED arrangement, and cause the capture of the first digital image by the top digital camera to determine a luster grade of the pearl by:
calculating a max luminance (Lumi_max) and a background luminance (Lumi_bg) from the first digital image;
locating a bright spot based on the Lumi_max and the Lumi_bg at a threshold of luminance at:
$$Th = ( Lumi\_max + Lumi\_bg ) * 0.5 ;$$
calculating a size of the bright spot based on boundary detection of pixels in the first digital image.

9. The system of claim 1, wherein the computer is configured to turn off the top LED arrangement, turn on the side LED arrangement, cause the capture of the first digital image by the top digital camera and apply a contrast filter to the first digital image to find three zones comprising: a boundary zone, a diffuse light zone, and a direct light zone, and analyze the three zones to determine body color and overtone, wherein the applying the contrast filter to find three zones includes:
creating a mask of the pearl as M-all;
creating a dark region as M-dark, a bright region as M-bright based on a histogram
creating an inner circle mask as M-inner based on the dark region; and
creating the three zones based on M-all, M-dark, M-bright, and M-inner, wherein the boundary zone is between M-all and M-inner, wherein the direct light zone is M-bright, wherein the diffuse light zone is M-dark.

10. The system of claim 9, wherein the computer is configured to, for white and light-colored pearls, analyze the boundary zone and the diffuse light zone for a body color grade, and analyze the direct light zone for a overtone grade.

11. The system of claim 9, when the pearl comprises a dark-colored pearl, the computer is configured to analyze the boundary zone and the direct light zone for an overtone grade, and the diffuse light zone for a body color grade.

12. The system of claim 9, wherein the computer is configured to perform a color analysis on the boundary zone, the diffuse light zone, and the direct light zone, for each zone calculate a mean value of Hue, by:
converting RGB to HSV on each pixel, H: hue in range 0°˜360°;
converting for each Hue value to (a*, b*) plane by:
$${a}^{*} = cos ( h )$$
$${b}^{*} = sin ( h )$$
calculating mean values a̅*̅ and b̅*̅;
calculating the mean value of Hue of each zone by:
$$\overline{h} = a tan ( \overline{{b}^{*}} / \overline{{a}^{*}} ) ;$$
and
evaluating the body color and overtone of the pearl based the mean values of Hue of each zone.

13. A method for imaging and grading a pearl, the method comprising:
sending, by a computer, a first instruction to a side light-emitting diode (LED) to cause illumination of a first side of the pearl with direct light from the side LED;
causing, by the computer, illumination of a second side of the pearl by a concave diffuser configured to diffuse light from the side LED on the second side of the pearl;
causing a capture of a first digital image of the pearl by a side digital camera;
receiving the first digital image from the side digital camera;
detecting a top surface of each pearl from the first digital image captured by the side digital camera;
aligning the top surface of each pearl to the side digital camera;
causing a capture of a second digital image of the pearl by a top digital camera, the second digital image including the pearl illuminated on the first side by the direct LED light and the diffused LED light on the second side;
receiving the second digital image from the top digital camera; and
calculating one or more characteristic grades for the pearl based on the received digital images from the top digital camera and side digital camera.

14. The method of claim 13, further comprising:
sending, by the computer, an instruction to motors on a stage configured to support the pearl, wherein the stage is configured with motors to move the stage in an X, Y, and Z direction.

15. The method of claim 13, further comprising:
determining, by the computer, any of a size grade and a roundness grade of the one or more characteristic grades for the pearl based on boundary detection of pixels in the received digital images.

16. The method of claim 13, further comprising:
determining, by the computer, any of a luster grade, a body color grade, and an overtone grade of the one or more characteristic grades for the pearl based on the received digital images.

17. The method of claim 13, further comprising:
turning off the side LED;
turning on a top LED; and
causing the capture of the second digital image by the top digital camera to determine a luster grade by calculating a max luminescence number displayed in the second digital image by:
calculating a max luminance (Lumi_max) and a background luminance (Lumi_bg) from the digital image;
locating a bright spot based on the Lumi_max and the Lumi_bg at a threshold of luminance at:
$$th = ( Lumi\_max + Lumi\_bg ) * 0.5 ;$$
calculating a size of the bright spot based on boundary detection of pixels in the second digital image.

18. The method of claim 13, further comprising:
turning off a top LED;
turning on the side LED; and
applying a contrast filter to the second digital image to find three zones: a boundary zone, a diffuse light zone, and a direct light zone, and analyzing the three zones to determine a body color and an overtone of the pearl, wherein the applying the contrast filter to find three zones includes:
creating a mask of the pearl as M-all;
creating a dark region as M-dark, a bright region as M-bright based on histogram analysis;
creating an inner circle mask M-inner based on dark region; and
creating 3 zones based on M-all, M-dark, M-bright, and M-inner, wherein the boundary zone is between M-all and M-inner, wherein the direct light zone is M-bright, and wherein the diffuse light zone is M-dark.

19. The method of claim 18, further comprising:
for white and light-colored pearls, analyzing, by the computer, the boundary zone and the diffuse light zone for a body color grade, and analyzing the direct light zone for an overtone grade; and
for dark-colored pearls, analyzing, by the computer, the boundary zone and the direct light zone for an overtone grade, and analyzing the diffuse light zone for a body color grade.

20. The method of claim 18, wherein a body color grade and an overtone grade is determined by color analysis on the boundary zone, the diffuse light zone and the direct light zone, for each zone calculating a mean value of Hue, by,
converting of RGB to HSV on each pixel, H: hue in range 0°˜360°;
converting for each Hue value to (a*, b*) plane by:
$${a}^{*} = cos ( h )$$
$${b}^{*} = sin ( h )$$
calculating mean values a̅*̅ and b̅*̅;
calculating the mean value of Hue of zone by:
$$\overline{h} = a tan ( \overline{{b}^{*}} / \overline{{a}^{*}} ) ;$$
and
evaluating the body color and the overtone of the pearl based the mean values of Hue for each zone.
